(19) United States
(12) Patent Application Publication
(10) Pub. No.: US 2011/0307041 A1
(43) Pub. Date: Dec. 15, 2011

(54) COOLING EYE MASK

(75) Inventor: RIC FLOYD, Waxhaw, NC (US)

(73) Assignee: FFPCo, LLC, Waxhaw, NC (US)

(21) Appl. No.: 13/157,121

(22) Filed: Jun. 9, 2011

Related U.S. Application Data
(60) Provisional application No. 61/353,079, filed on Jun. 9, 2010.

(51) Int. Cl. A61F 7/10; C08L 29/04

(52) U.S. Cl. 607/109; 523/105

(57) ABSTRACT

An eye pack for evaporative cooling of the face proximate to one or more eyes is provided. In one aspect, an eye pack is disclosed comprising a gelatinous foam impregnated with an aqueous solution of polyvinyl alcohol embedded therein, and a barrier affixed to at least one side of the foam, wherein the barrier allows transfer of heat and restricts the transfer of the solution. In another aspect, a cooling eye pack, includes a gelatinous material including glycerin, a hydrophillic polymer, water, at least one paraben, and any one or more of ethylenediaminetetraacetic acid tetrasodium salt and ethylenediaminetetraacetic acid disodium salt; and a head securement adapted to secure the eye pack to a face proximate to one or more eyes of a human.

### FIELD

The present disclosure relates to an eye patch or eye packs for soothing and/or cooling one or more eyes and proximate facial areas. In one aspect, the disclosure relates to evaporative cooling based eye packs that avoid the need for preparatory refrigeration. In another aspect, the disclosure relates to a disposable and/or recyclable eye pack that is made from biodegradable and/or recyclable material. In a still further aspect, the disclosure relates to a safe and effective method of cooling the face proximate and including one or more eyes utilizing an evaporative cooling material and/or similar chemical coolant.

### BACKGROUND

Asthenopia or eye strain occurs when eyes get tired from intense use, such as driving a car for extended periods, reading, or working at a computer. Continuously focusing on a book or computer monitor often causes the ciliary muscles to tighten, thereby causing the eyes to get irritated and uncomfortable. It is estimated that each year about 10 million people seek eye exams for computer-related eyestrain. Allergies can create sensitivities, irritation, and redness in eyes in response to environmental conditions. The cooling of muscles to relieve tension and stress is well known in the art, but the eyes pose additional problems as chemical based solutions can cause greater irritation and possibly even temporary or permanent blindness.

Solutions in the art for irritated, swollen, strained, puffy, and/or tired eyes have generally been limited to pre-refrigerated eye packs that are not easily portable and don't allow much freedom of movement while applied. Such cooling packs are intended to alleviate all the above symptoms in addition to reducing tension in the ciliary muscles and providing additional beneficial effects to the eye lids and areas around the eyes. A majority of such eye packs include plastic or vinyl enclosures fitted for the eyes and contain some coolant sealed therein that necessitates some form of preparatory refrigeration. While refrigerated cooling packs are a natural solution for soothing strained eyes, existing cooling packs require preparatory refrigeration, are limited as a result regarding their portability and use while not near the means to refrigerate the packs, and do not allow the user much freedom of movement (i.e., a user must be laying down and/or sitting back and still). Non-refrigerated chemical based cooling compresses have been known in the art for other areas of the body, but utilize a compression effect which would not be applicable to the sensitive area near the eyes. Such compresses may also utilize harmful chemicals not suitable for the eyes.

Likewise and as mentioned above, the use of chemicals or chemical based packs is limited since most cooling chemicals are irritants to the eyes. For instance, menthol is a common ingredient in cooling muscle salves, including one manufactured by Avon® (discontinued), containing 5% menthol for soothing sore muscles. If menthol comes into contact with the eye, however, it produces a burning sensation lasting at least 15-30 minutes. This is counter to the goals of the present disclosure. In consequence, the art continues to seek improvements in portable and/or disposable eye packs that avoid the need for preparatory refrigeration.

### SUMMARY

The present disclosure relates to an eye pack that avoids the need for preparatory refrigeration.

In one aspect, the disclosure relates to a disposable and/or recyclable eye pack made from biodegradable and/or recyclable material, respectively.

In another aspect, the disclosure relates to a safe and effective method of cooling one or more area proximate and including one or more eyes utilizing an evaporative cooling material, composition, and/or chemical coolant that is not an irritant to the eyes.

In a still further aspect, a securing apparatus such as a non-permanent adhesive that is generally not an irritant to the skin is provided to allow the eye pack to be placed over the eyes without the discomfort of compression. The non-permanent adhesive is used to temporarily secure the eye pack to the face of a user proximate the one or more eyes, wherein the user should experience no discomfort from any pressure on the eyes and is allowed greater freedom of movement than would otherwise be practical absent the securing apparatus. Additionally, the securing apparatus would provide a sealing effect of the cooling pack, thus increasing the effectiveness and duration of the cooling effect in one aspect.

In a further aspect, the disclosure relates to a cooling eye pack, comprising: a gelatinous foam impregnated with a coolingly effective amount of an aqueous solution of polyvinyl alcohol embedded therein; and a barrier affixed to at least a portion of one side of the foam, wherein the barrier allows transfer of heat and restricts the passage of the solution.

A further aspect of the disclosure relates to a method for cooling a facial eye region, comprising: securing an eye pack to a facial eye region, the eye pack further comprising: a gelatinous material including glycerin, a hydrophillic polymer, water, at least one paraben, and any one or more of ethylenediaminetetraacetic acid tetrasodium salt and ethylenediaminetetraacetic acid disodium salt; and a head securement adapted to secure the eye pack to a face proximate to one or more eyes of a human.

A still further aspect of the disclosure relates to a cooling eye pack, comprising: a gelatinous material including glycerin, a hydrophillic polymer, water, at least one paraben, and any one or more of ethylenediaminetetraacetic acid tetrasodium salt and ethylenediaminetetraacetic acid disodium salt; and a head securement adapted to secure the eye pack to a face proximate to one or more eyes of a human.

Other aspects, features and embodiments of the disclosure will be more fully apparent from the ensuing disclosure and appended claims.

### BRIEF DESCRIPTION OF THE DRAWINGS

FIG. 1 is a schematic view of the eye pack in one embodiment of the present disclosure with a cut-away fold revealing the underlying material beneath the barrier.

FIG. 2 is a schematic view of the eye pack in one embodiment with a securing apparatus in the form of adhesive sections.

### DETAILED DESCRIPTION AND PREFERRED EMBODIMENTS

The disclosure, as variously described herein in respect of features, aspects and embodiments thereof, may in particular implementations be constituted as comprising, consisting, or consisting essentially of, some or all of such features, aspects and embodiments, as well as elements and components thereof being aggregated to constitute various further implementations of the disclosure. The disclosure is described herein in various embodiments, and with reference to various features and aspects of the disclosure. The disclosure contemplates such features, aspects and embodiments in various permutations and combinations, as being within the scope of the disclosure. The disclosure may therefore be specified as comprising, consisting, or consisting essentially of, any of such combinations and permutations of these specific features, aspects and embodiments, or a selected one or ones thereof.

The present disclosure relates to an eye pack for one or more eyes. In one embodiment, the eye pack comprises an absorbent, foam or the like, material capable of retaining a solution of polyvinyl alcohol (PVA) and/or other related chemicals that are not eye irritants in diluted percentages. In various other embodiments, the weight percent PVA in the aqueous polyvinyl alcohol solution may be in a range having a lower value of any of 1%, 2%, 3%, 4%, 5%, 6%, 7% and 8%, and having an upper value of any of 5%, 6%, 7%, 8%, 9%, 10%, 11%, 12%, 13%, 14%, 15%, 16%, 17%, 18%, 19% and 20%, wherein the weight percentage is based on the total weight of the solution, and subject to the proviso that any of the lower limit values specified in this sentence may define a range whose upper value is any of the upper values specified in this sentence that is larger in magnitude than the lower limit value.

The present disclosure also relates to an eye pack comprising a gelatinous material including glycerin, a hydrophillic polymer, water, at least one paraben, and any one or more of ethylenediaminetetraacetic acid tetrasodium salt and ethylenediaminetetraacetic acid disodium salt.

The disclosed eye pack solutions and gelatinous material, when exposed to the heat radiating from a human body, evaporates thereby causing an evaporative cooling effect on the human. In one embodiment, the evaporative cooling effect absolves the need for preparatory refrigeration of the eye pack by way of a safe solution of polyvinyl alcohol or which has known attributes in medical eye drops at approximately 1.4%, yet also provides an adequate evaporative chemical for promotion of evaporative cooling of the eyes. Any similar chemical capable of being embedded in a foam, safe for the eyes, and having evaporative cooling qualities at room temperature may be supplemented for polyvinyl alcohol.

As an alternative or addition to polyvinyl alcohol, an eye pack in one embodiment comprises a safe solution of glycerin, a hydrophillic polymer, water, at least one paraben, and any one or more of ethylenediaminetetraacetic acid tetrasodium salt and ethylenediaminetetraacetic acid disodium salt.

In one embodiment, gelatinous foam or material is adapted to be impregnated with an aqueous solution of polyvinyl alcohol or solution of glycerin, a hydrophillic polymer, water, at least one paraben, and any one or more of ethylenediaminetetraacetic acid tetrasodium salt and ethylenediaminetetraacetic acid disodium salt, that includes a barrier on at least the side that would be exposed to the skin and eyelids and eye sockets of the face to further insure against exposure of the evaporative chemicals to the eyes.

The term “barrier” as used herein may include any material sufficient to preclude the transfer of solution in one direction towards the eyes, but yet allows the transfer of heat in the opposite direction from the eyes towards the foam and exterior, away from the eyes. In one additional embodiment, the foam may be a synthetic resin (polyurethane), wherein the polyvinyl alcohol may be combined with glycerine, glycerol and/or a borax solution to form the gel and/or to aid in impregnating the solution into the foam. Other well known compounds in the art may be provided to thicken the gelatinous material.

In a one embodiment, the eye pack may include foam or absorbent material that is preferably disposable and/or biodegradable. Totally biodegradable foam is not truly “plastic” but is made from renewable and sustainable resources such as natural starch and/or cellulose. Examples of such biodegradable foam include Green Cell Foam™ and/or foam made from corn starch, natural starch, cellulose, and/or a combination thereof. In one embodiment, the eye pack may comprise a waterlock material including any one of more of polyacrylic resin and sodium polyacrylate.

The barrier in one embodiment would be disposable and/or biodegradable. Packaging in various embodiments would also ideally be disposable and/or biodegradable, but yet would sufficiently seal the contents and preserve an acceptable shelf life for retaining the gelatinous foam or material to limit the same from evaporating.

In another aspect, a cooling eye pack, includes a gelatinous material including glycerin, a hydrophillic polymer, water, at least one paraben, and any one or more of ethylenediaminetetraacetic acid tetrasodium salt and ethylenediaminetetraacetic acid disodium salt, and a head securement adapted to secure the eye pack to a face proximate to one or more eyes of a human.

In one embodiment, water comprises at least 55% or more of the gelatinous material. In one embodiment, the water comprises from about 55% to about 86% by weight of the gelatinous material. In an alternative embodiment, an alcohol may displace any volume of water. In one embodiment, an alcohol comprises from about 0.1% to about 5% weight of the gelatinous material, which may be in addition to or in place of that same percentage of water. In one embodiment, the alcohol may comprise any one or more of ethanol, polyvinyl alcohol, or any other type of alcohol in such percentages that is not harmful to the eyes or skin of most humans.

In various embodiments, glycerin comprises from about 20% to about 30% by weight of the gelatinous material. In various embodiments, the hydrophillic polymer comprises a waterlock material including any one of more of polyacrylic resin and sodium polyacrylate, wherein the any one of more of polyacrylic resin and sodium polyacrylate comprises from about 3% to about 10% by weight of the gelatinous material.

In various embodiments, the at least one paraben comprises any one or more of methylparaben and propylparaben from about 0.1% to about 0.5% by weight of the gelatinous material. In various embodiments, the any one or more of ethylenediaminetetraacetic acid tetrasodium salt and ethylenediaminetetraacetic acid disodium salt comprises from about 0.1% to about 0.5% by weight of the gelatinous material.

The gelatinous material includes in various embodiments any desired thickness necessary to retain the gelatinous material in a cooling state for approximately six to approximately eight hours. In one embodiment, the gelatinous material includes a thickness of about 0.5 mm to about 2 mm.

The eye pack in various embodiments may further comprise any one or more of moisturizers, skin repairers, essential oils and minerals, vitamins, and aromatic scents.

In a further aspect, a method for cooling a facial eye region includes securing an eye pack to a facial eye region, the eye pack comprising a gelatinous material including glycerin, a hydrophillic polymer, water, at least one paraben, and any one or more of ethylenediaminetetraacetic acid tetrasodium salt and ethylenediaminetetraacetic acid disodium salt, and a head securement adapted to secure the eye pack to a face proximate to one or more eyes of a human. The method provides for cooling of a facial eye region for at least six to eight hours and will continue to have a cooling effect for as long as the pack stays in place on a user's face and there is gelatinous material remaining In addition, the disclosed eye pack can be reused 4 or 5 times if stored in a sealed bag or container. Preservation of the disclosed eye packs is best at a temperature range of from about 0° F. to about 80° F., and more preferably from about 40° F. to about 70° F.

The advantages and features of the disclosure are further illustrated with reference to the following examples, which are not to be construed as in any way limiting the scope of the disclosure but rather as illustrative of various embodiments of the invention in specific applications thereof.

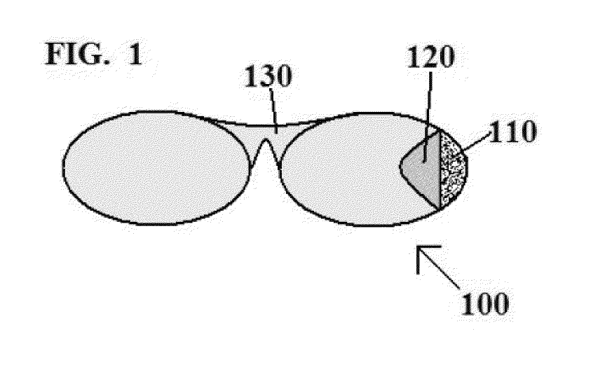

FIG. 1 depicts an eye pack 100 in one embodiment of the present disclosure with a cut-away fold of optional barrier material 120 revealing the underlying foam material 110 beneath the bather 120.

The foam material 110 in one embodiment is impregnated with an aqueous solution of polyvinyl alcohol, preferably from about 4% to 10% by weight, and more preferably from about 6% to 8% by weight. The barrier 120 allows the transfer of heat from the face to accelerate the evaporation of the polyvinyl alcohol from the foam material 110 thereby effectuating an evaporative cooling effect on the face of the user. The optional barrier 120 may be adapted to a portion of one side of the eye pack 100, allowing the exterior side opposite the face to be exposed to the open air after removing the eye pack 100 from its packaging (not shown) to further effectuate the evaporative cooling effect. The entire eye pack 100 could be encased in the barrier 120, however, the barrier side opposite the face would benefit from vent holes (not shown) or from a material that allows the exposure of solution to ambient air to permit evaporation of the same. One preferred barrier comprises a hydrophobic foam known in the art, such as that disclosed in U.S. Pat. No. 6,747,068, incorporated herein by reference.

The material 110 in an alternative embodiment comprises a gelatinous material comprising glycerin, a hydrophillic polymer, water, at least one paraben, and any one or more of ethylenediaminetetraacetic acid tetrasodium salt and ethylenediaminetetraacetic acid disodium salt. A barrier 120 may or may not be necessary in such alternative embodiment.

The water comprises from about 55% to about 86% by weight of the gelatinous material 110. An alcohol may be used in place of any volume of water, wherein alcohol comprises from about 0.1% to about 5% weight of the gelatinous material 110 in one embodiment. In various embodiments, the alcohol may comprise any one or more of ethanol and polyvinyl alcohol. Glycerin comprises from about 20% to about 30% by weight of the gelatinous material 110. The hydrophillic polymer comprises a waterlock material including any one of more of polyacrylic resin and sodium polyacrylate, wherein the any one of more of polyacrylic resin and sodium polyacrylate comprises from about 3% to about 10% by weight of the gelatinous material 110. The at least one paraben comprises any one or more of methylparaben and propylparaben from about 0.1% to about 0.5% by weight of the gelatinous material 110. The any one or more of ethylenediaminetetraacetic acid tetrasodium salt and ethylenediaminetetraacetic acid disodium salt comprises from about 0.1% to about 0.5% by weight of the gelatinous material 110.

The gelatinous material 110 includes in various embodiments any desired thickness necessary to retain the gelatinous material 110 in a cooling state for approximately six to approximately eight hours. In one embodiment, the gelatinous material 110 includes a thickness of about 0.5 mm to about 2 mm.

Additionally, the eye pack 100 can take many shapes and forms, in addition to including an impregnated foam solution in the area approximate to the bridge of a user's nose relative to the nose piece 130 of the eye pack 100. Alternatively, the nose piece 130 of the eye pack 100 could act as a handle for applying and removing the eye pack 100 from a user's face. Finally, the nose piece 130 could optionally include a handle on the exterior and foam 110 with or without a barrier 120 on the interior.

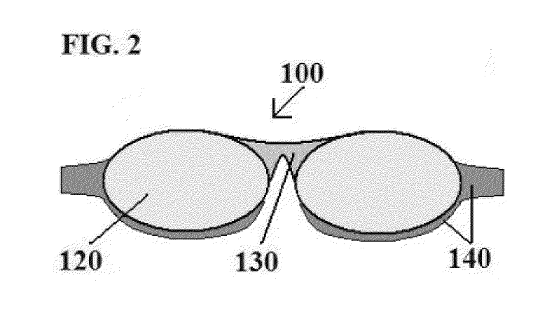

An eye pack 100 of the type shown schematically in FIG. 2 is employed with an optional temporary adhesive 140 for securing to the face of a user. In a preferred embodiment, an adhesive 140 coupled with the eye pack 100 adheres the pack 100 to the facial area below and to the side of the eyes to effectuate a sealing of the pack 100 and cooling effect proximate and including the eyes, thereby enhancing the cooling effectiveness and duration of cooling effect. The adhesive 140 may also be optionally included in the area around the bridge of the nose 130. The adhesive 140 additionally allows the pack 100 to be applied with little or no compression of the eyes and is fully portable and useable in any body position, including but not limited to seated, reclined, on an air plane, and sleeping, in addition to face up or down. Locating the adhesive 140 towards each end of the pack 100 allows for compression of the area immediately to the outer edges of each eye in a manner that would reduce wrinkles commonly known as “crow's feet.” As intended, the cooling effect also reduces irritation and swelling around the eyes, the eye lids and below the eyes, thereby reducing redness and puffiness.

In a further embodiment, the foam or gelatinous material could be infused with scented oils in the area around the nose 130 such as lavender, a known sleep aid, peppermint (a known nasal decongestant), and/or cucumber (a vegetable commonly used in cooling the eyes). In yet a further embodiment, the or gelatinous foam material could be easily shaped to better fit the face and eyes of the user by providing the foam in a thin layer that is easily cut by scissors or the like. In still another embodiment, the eye pack 100 could have an attractive design, pattern or advertisement printed on the exterior.

In a separate embodiment, the packaging for the eye pack 100 would ideally be adapted to seal the pack 100 and possibly even have a re-sealable opening to allow re-use of the pack 100. Such a container might ideally be made of any non-rigid plastic known in the art and capable of prolonging the shelf life of the pack 100.

While the disclosure has been described herein in reference to specific aspects, features and illustrative embodiments of the disclosure, it will be appreciated that the utility of the disclosure is not thus limited, but rather extends to and encompasses numerous other variations, modifications and alternative embodiments, as will suggest themselves to those of ordinary skill in the field of the present invention, based on the disclosure herein. It is to be appreciated that any of the elements and features described herein may be combined with any one or more other elements and features. Correspondingly, the disclosure as hereinafter claimed is intended to be broadly construed and interpreted, as including all such variations, modifications and alternative embodiments, within its spirit and scope.

What is claimed is:

1. A cooling eye pack, comprising:
a gelatinous foam impregnated with a coolingly effective amount of an aqueous solution of polyvinyl alcohol embedded therein;
wherein the coolingly effective amount of aqueous solution comprises about 4% to 10% by weight of the polyvinyl alcohol.

2. A method for cooling a facial eye region, comprising:
securing an eye pack to a facial eye region, the eye pack comprising:
a gelatinous material including glycerin, a hydrophillic polymer, water, at least one paraben, and any one or more of ethylenediaminetetraacetic acid tetrasodium salt and ethylenediaminetetraacetic acid disodium salt; and
a head securement adapted to secure the eye pack to a face proximate to one or more eyes of a human.

3. The method of claim 2, wherein glycerin comprises from about 20% to about 30% by weight of the gelatinous material.

4. The method of claim 2, wherein the hydrophillic polymer comprises a waterlock material including any one of more of polyacrylic resin and sodium polyacrylate.

5. The method of claim 4, wherein the any one or more of polyacrylic resin and sodium polyacrylate comprises from about 3% to about 10% by weight of the gelatinous material.

6. The method of claim 2, wherein the at least one paraben comprises any one or more of methylparaben and propylparaben from about 0.1% to about 0.5% by weight of the gelatinous material.

7. The method of claim 2, wherein the any one or more of ethylenediaminetetraacetic acid tetrasodium salt and ethylenediaminetetraacetic acid disodium salt comprises from about 0.1% to about 0.5% by weight of the gelatinous material.

8. The method of claim 2, wherein the gelatinous material includes a thickness of about 0.5 mm to about 2 mm.

9. The method of claim 2, wherein the water comprises from about 55% to about 86% by weight of the gelatinous material.

10. The method of claim 2, wherein gelatinous material further includes an alcohol comprising from about 0.1% to about 5%.

11. The method of claim 2, wherein gelatinous material further includes any one or more of essential oils and minerals, vitamins, and aromatic scents.

12. The method of claim 2, wherein the head securement comprises a temporary adhesive compatible with human skin.

13. A cooling eye pack, comprising:
a gelatinous material including glycerin, a hydrophillic polymer, water, at least one paraben, and any one or more of ethylenediaminetetraacetic acid tetrasodium salt and ethylenediaminetetraacetic acid disodium salt; and
a head securement adapted to secure the eye pack to a face proximate to one or more eyes of a human.

14. The eye pack of claim 13, wherein glycerin comprises from about 20% to about 30% by weight of the gelatinous material.

15. The eye pack of claim 13, wherein the hydrophillic polymer comprises a waterlock material including any one of more of polyacrylic resin and sodium polyacrylate.

16. The eye pack of claim 15, wherein the any one or more of polyacrylic resin and sodium polyacrylate comprises from about 3% to about 10% by weight of the gelatinous material.

17. The eye pack of claim 13, wherein the at least one paraben comprises any one or more of methylparaben and propylparaben from about 0.1% to about 0.5% by weight of the gelatinous material.

18. The eye pack of claim 13, wherein the any one or more of ethylenediaminetetraacetic acid tetrasodium salt and ethylenediaminetetraacetic acid disodium salt comprises from about 0.1% to about 0.5% by weight of the gelatinous material.

19. The eye pack of claim 13, wherein the gelatinous material includes a thickness of about 0.5 mm to about 2 mm.

20. The eye pack of claim 13, wherein the water comprises from about 55% to about 86% by weight of the gelatinous material.

21. The eye pack of claim 13, wherein gelatinous material further includes an alcohol comprising from about 0.1% to about 5%.

22. The eye pack of claim 13, wherein gelatinous material further includes any one or more of essential oils and minerals, vitamins, and aromatic scents.

23. The eye pack of claim 13, wherein the head securement comprises a temporary adhesive compatible with human skin.
